(19) United States
(12) Patent Application Publication
(10) Pub. No.: US 2024/0074663 A1
(43) Pub. Date: Mar. 7, 2024

(54) MECHANICAL MEASURING DEVICE

(71) Applicants: HIROSHIMA UNIVERSITY, Higashi-hiroshima-shi, Hiroshima (JP); TAKUMI Co., LTD.,, Yao-shi, Osaka (JP)

(72) Inventors: Nobuo ADACHI, Higashi-hiroshima-shi (JP); Masakazu ISHIKAWA, Higashi-hiroshima-shi (JP); Goki KAMEI, Higashi-hiroshima-shi (JP); Kenzo SUZUKI, Yao-shi (JP); Shigehisa SUZUKI, Yao-shi (JP)

(73) Assignees: HIROSHIMA UNIVERSITY, Higashi-hiroshima-shi, Hiroshima (JP); TAKUMI Co., LTD.,, Yao-shi, Osaka (JP)

(21) Appl. No.: 18/272,686

(22) PCT Filed: Jan. 17, 2022
(86) PCT No.: PCT/JP2022/001307

§ 371 Date: Jul. 17, 2023

(30) Foreign Application Priority Data
Jan. 18, 2021 (JP) 2021-005442

(51) Int. Cl. A61B 5/00; A61B 5/107

(52) U.S. Cl. CPC A61B 5/0053; A61B 5/1072; A61B 2090/064

(57) ABSTRACT

A mechanical measuring device includes a body case, a probe 10 extending from the body case and abutting against a predetermined portion of a biological soft tissue, a load measuring means 12 which is integrally connected to the probe 10 and which measures a load applied to the biological soft tissue by the probe 10, a moving means 14 for moving the probe 10 at a constant speed, and a distance measuring means 20 for measuring a moving distance by the moving means 14, and in a measuring operation of the biological soft tissue such as muscle and cartilage, the load applied to the biological soft tissue and the moving distance of the probe 10 are simultaneously measured in a state where the probe 10 is moved at a constant speed, thereby realizing inspection and diagnose of a mechanical state of the biological soft tissue.

### TECHNICAL FIELD

The present invention relates to a mechanical measuring device capable of measuring mechanical characteristic of a biological soft tissue when pressing force or tensile force is applied to the biological soft tissue such as ligament, muscle and cartilage, and capable of quantitatively and objectively inspect and diagnose a state of the biological soft tissue.

### BACKGROUND TECHNIQUE

In a surgical operation such as arthroscopic surgery, a doctor usually abuts a rod-like stainless probe on a predetermined portion of a biological soft tissue, and the doctor qualitatively inspects and diagnoses a damaged state of the biological soft tissue by his or her own judgment while relying on senses of force transmitted from the abutted portion to his or her hand through the probe. These inspection and diagnosis are based on subjective finding of the doctor, and the inspection and the diagnosis are largely influenced by knowledge and experience of the doctor. As described above, in the conventional technique, unified inspection and diagnose based on objective reference are not carried out, and this is real circumstance that inspection and diagnose of a probe carried out by an inexperienced doctor are especially difficult. Therefore, there are many requests that inspection and diagnose of a probe are carried out quantitatively and objectively.

Here, the biomedical tissue is classified into a biological hard tissue such as bone and tooth, and into a biological soft tissue such as muscle, ligament, and cartilage. For example, as a technique for quantitatively inspecting and diagnosing the biological soft tissue, there are patent documents 1 to 3, and as a technique for quantitatively inspecting and diagnosing the biological hard tissue, there is patent document 4.

Patent document 1 shows an inspecting and diagnosing device of joint by using a probe. A stress detector detects force applied to a tip end of the probe (see FIGS. 1 and 2 of patent document 1). Further, patent document 1 shows a state where a user latches articular lip which is a biological soft tissue on a tip end of the probe, and the articular lip is pulled by the probe (see FIG. 6(a) of patent document 1), and shows a state where the user presses the tip end of the probe against cartilage which is the biological soft tissue (see FIG. 6(b) of patent document 1). As described above, patent document 1 discloses a technique to measure force applied to the tip end of the probe, and to quantitatively and objectively inspect and diagnose a state of the biological soft tissue.

Patent document 2 measures tensile force of tendon or ligament. An extension amount of a hook shaft is measured by a tensile force spring which is previously set to certain tensile force. In this case, to measure the extension amount, a spike portion of abutting means is abutted against the bone, a surface of the abutted bone is set at a reference position for measuring the extension amount, and the extension amount on which tensile force actually acts is obtained from a difference between the extension amount of the probe and a moving distance of the abutting means.

Patent document 3 quantitatively detects mechanical characteristic of ligament. Patent document 3 shows an example in which a probe is moved manually, a load is detected by a strain gauge, and displacement quantity is detected by displacement quantity of rules defined utilizing spring force (see FIG. 1 of patent document 3).

On the other hand, patent document 4 discloses a method and a device for evaluating strength of bones of human and animal which are biological hard tissues. Patent document 4 shows a rotating horizontal cam which interlocks with rotation of a motor, and a displacement generator composed of a follower pin which vertically moves as the cam rotates (see FIGS. 3A and 3B of patent document 4). This follower pin has such a configuration in which the inspection probe moves vertically, and patent document 4 shows a configuration of a force sensor capable of detecting force applied to the inspection probe, and a configuration of a distance sensor capable of detecting a moving distance of the force. Patent document 4 shows a configuration in which a reference probe is provided in addition to the inspection probe, and an inserting degree of the inspection probe is specified by the reference probe (see FIGS. 1A to 1C of patent document 4). As described above, patent document 4 discloses a technique in which a state of a biological hard tissue is quantitatively and objectively inspected and diagnosed.

### PRIOR ART DOCUMENTS

### Patent Documents

[Patent Document 1] Japanese Patent No. 6253045
[Patent Document 2] Japanese Patent Application Laid-open No. 2002-39882
[Patent Document 3] Japanese Patent Application Laid-open No. 2000-201906
[Patent Document 4] Japanese Patent No. 4918086

### SUMMARY OF THE INVENTION

### Object to be Solved by the Invention

The biological soft tissue can be regarded as viscoelastic body. The viscoelastic body has characteristic that it is soft mechanically, and speed dependency exists between stress and distortion. When a state of such a biological soft tissue is mechanically inspected and diagnosed, it is necessary to measure two values: force applied to the biological soft tissue and a distance through which this force is applied. However, at this time, it is absolutely necessary to take the following two points into consideration. The first point is that there exists speed dependency between force σ (stress) applied to the biological soft tissue and a distance ε (distortion) through which this force is applied. When a speed of the applied force σ is large, as compared with a case where the force is small, the force σ appears large as compared with the distance (distortion) e through which this force is applied. When force is applied to the same measured objects with a different speed, a relation between the force and the distance at that time shows a different result. Therefore, if a speed of the applied force is varied during measuring operation, relation between the force σ and the distance (distortion) e cannot precisely be measured. Hence, in order to precisely measure the mechanical characteristic of the biological soft tissue which is viscoelastic body, it is an absolutely necessary condition to make the speed of the force applied to the biological soft tissue constant.

The second point is that unlike a rigid body, force σ/distance ε characteristic (rigidity) is not always linear but nonlinear. Hence, it is necessary to measure rigidity (stiffness) at respective points or regions, and it is necessary to measure (measure in real time) the rigidity (stiffness) while associating a moving distance of probe and force at the point or region.

According to the configuration shown in patent document 1, the state of the biological soft tissue is quantitatively inspected and diagnosed, but the probe is moved manually (using thumb), and it is not possible to constantly keep the moving speed. The moving distance is detected by visually reading scale carved on a device, and it is impossible to simultaneously and precisely measure the detection of the moving distance and the detection of force at that point or region, and time difference is generated between detection of the moving distance and detection of the force at any price. Therefore, it is not possible to successively detects both of them, and to measure in real time. Further, in order to detect the moving distance, a reference point for detecting a distance is normally necessary, but patent document 1 does not describe the setting of the reference point. Hence, instant when a tip end of the probe abuts against the measured object is visually checked, a position of the probe at that time is regarded as the reference point, and the moving distance is detected. However, ligament or cartilage of a biological body exists in the biological body, a small hole is formed in the biological body, the probe is inserted into the hole, and the probe is abutted against the ligament or the cartilage. It is necessary to take a peek of the ligament or cartilage through the hole and to visually check a contact point between the probe and the measured object, and it is impossible to find out the contact point precisely. Therefore, it is impossible to precisely set the reference point, and the moving distance detected becomes inaccurate.

Like patent document 1, patent document 2 also moves the device manually and measures. Hence, a moving speed of acting force is not constant, and it is impossible to measure at a constant speed. Further, acting force is measured by visually carrying out acting force scale, and the moving distance of the probe (i.e. extension amount) is measured by visually carrying out moving scale. Therefore, it is impossible to simultaneously measure the acting force and the extension amount.

In patent document 3 also, measurement is carried out manually. Therefore, the moving speed of a load (force) is not constant, and it is impossible to measure at the constant speed. Although the load is automatically measured by a strain gauge, moving distance of the probe (i.e. displacement quantity) is continuously measured, and it is impossible to measure displacement quantity and load one-on-one successively in real time. In patent document 3, there is no setting of the reference point for detecting the moving distance. Hence, like patent document 1, since abutment between the tip end of the probe and the measured object is visually checked and its abutment position is defined as the reference point, detected moving distance is not accurate.

On the other hand, configuration shown in patent document 4 relates to inspecting and diagnosing of a biological hard tissue, and patent document 4 is different from the present invention which inspects and diagnoses a biological soft tissue. In patent document 4, a rotating tilting cam interlocks with rotation of a motor and a follower pin vertically carries out reciprocal motion as this cam rotates. Therefore, a speed of the vertical reciprocating motion of the follower pin is not constant. Since the inspection probe vertically reciprocates in conjunction with the follower pin, the inspection probe cannot reciprocate at a constant speed either. In order to measure the moving distance of the inspection probe, it is usually necessary to set the reference position and thus, the reference probe is indispensable in patent document 4. The reference probe is abutted against a hard material (e.g. bone) which can withstand its pressing force, and the moving distance of the inspection probe is measured from a difference between the reciprocating inspection probe and a position of the fixed reference probe. Therefore, in this case, a hard material for fixing the reference probe is required, and measurement cannot be carried out by measured object made of only soft tissue such as viscoelastic body.

Hence, according to the present invention, a probe is moved at a constant speed in a biological soft tissue, pressing force or tensile force of the probe and a moving distance of the probe are simultaneously measured, thereby making it possible to precisely, quantitatively and objectively inspect and diagnose a state of the biological soft tissue.

### Means for Solving the Problem

A first mechanical measuring device of the present invention including a body case, a probe extending from the body case and abutting against a predetermined portion of a biological soft tissue, a load measuring means which is integrally connected to the probe and which measures a load applied to the biological soft tissue, a moving means for moving the probe at a constant speed, and a distance measuring means for measuring a moving distance by the moving means, wherein the load applied to the biological soft tissue and the moving distance of the probe are simultaneously measured.

In a second mechanical measuring device of the invention according to the first device, the device further includes rigidity calculating means for calculating rigidity (force/distance) of the biological soft tissue using output of the load measuring means and output of the distance measuring means.

According to a third mechanical measuring device of the invention, in the first or second device, a measuring operation of the moving distance of the probe is started from point in time when the load measuring means starts measuring the load applied to the biological soft tissue.

According to a fourth mechanical measuring device of the invention, in any one of the first to third devices, the constant speed by the moving means is appropriately changed.

According to a fifth mechanical measuring device of the invention, in any one of the first to fourth devices, the device further includes display means for displaying output of the load measuring means, output of the distance measuring means and output of the rigidity calculating means, and recording means for recording the outputs.

According to a sixth mechanical measuring device of the invention, in any one of the first to fifth devices, the device further includes supporting means for fixing a position of the body case.

According to a seventh mechanical measuring device of the invention, in any one of the first to sixth devices, the probe is made of translucent material, and scattering light is emitted from one end of the probe.

### Effect of the Invention

According to an embodiment of the present invention, in a measuring operation of a biological soft tissue such as ligament, muscle and cartilage, it is possible to simultaneously measure a load (force) applied to the biological soft tissue and a moving distance of a probe in a state where the probe is moved at a constant speed. Therefore, it is possible to precisely, quantitatively and objectively measure a state of the biological soft tissue which is subjected to inspection and diagnosis.

### BRIEF DESCRIPTION OF THE DRAWINGS

FIG. 1(a) is a sectional view of a mechanical measuring device according to a first embodiment, FIG. 1(b) is a top view of a supporting member 25, and FIG. 1(c) is a side view of the supporting member 25;

FIG. 2 is a model diagram of a biological soft tissue;

FIG. 3 are diagrams showing a shape of one end 10a of a probe 10;

FIG. 4(a) is a diagram showing a positional relation between the probe 10 and a ligament before and after a measuring operation is started, and FIG. 4(b) is a diagram showing a relation between a moving distance and a tensile force of the probe 10 when the ligament is measured;

FIG. 5 is a schematic diagram showing the mechanical measuring device and its circuit configuration relation of the first embodiment;

FIG. 6 is a diagram showing the circuit configuration relation of the first embodiment;

FIG. 7 is a diagram showing a relation between distortion, stress and rigidity of the biological soft tissue;

FIG. 8 is a schematic diagram showing a relation between force and a distance when the force is applied to the same biological soft tissue at a different speed;

FIG. 9 is a diagram showing variation of rigidity in characteristics of stress-distortion;

FIG. 10 is a diagram showing display output at display means 22b in the first embodiment;

FIG. 11 is a sectional view of a mechanical measuring device according to a second embodiment;

FIG. 12 is a schematic diagram showing the mechanical measuring device of the second embodiment and a circuit configuration relation thereof;

FIG. 13 is a perspective view of the mechanical measuring device of the second embodiment;

FIG. 14 are sectional views of configuration of essential portions of a mechanical measuring device of a third embodiment; and

FIG. 15 are explanatory diagrams of a supporting member of a mechanical measuring device of a fourth embodiment.

### MODE FOR CARRYING OUT THE INVENTION

Preferred embodiments of the present invention will be described below with reference to drawings.

### First Embodiment

### Configuration of a Mechanical Measuring Device

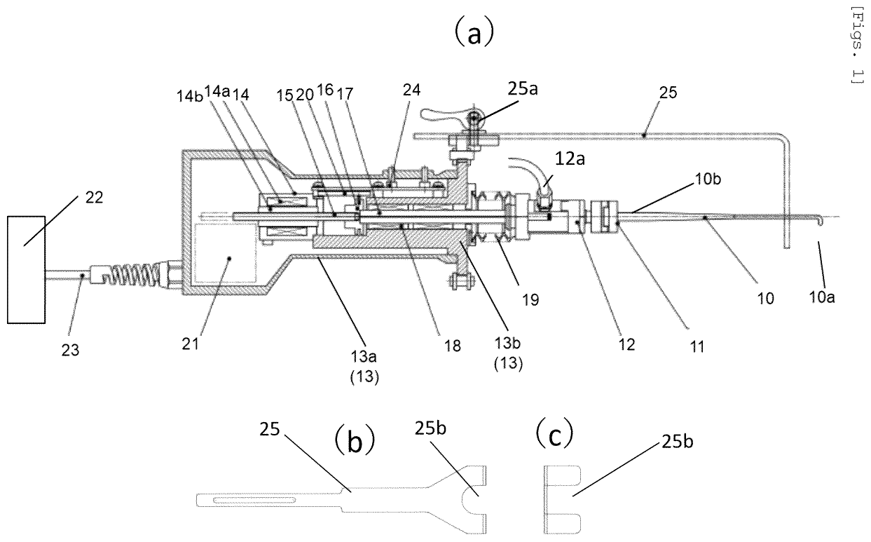

FIG. 1(a) is a sectional view of a mechanical measuring device according to a first embodiment. In FIG. 1(a), 10 represents a probe, 10a represents one end of the probe 10, 10b represents the other end, 11 represents a probe holder, 12 represents a load sensor, 12a represents an output terminal of the load sensor 12, 13 represents a body case, 13a represents a grasping portion of the body case 13, 13b represents a lid of the body case 13, 14 represents a stepping motor, 14a represents a motor coil, 14b represents a nut, 15 represents a screw shaft, 16 represents a connecting member, 17 represents a spline shaft, 18 represents a ball spline, 19 represents a cover, 20 represents a position detector, 21 represents an electronic circuit, 22 represents a controller, 23 represents an external cable, 24 represents an operation switch, 25 represents a supporting member, and 25a represents a fixing screw. FIGS. 1(b) and 1(c) are a top view and a side view of the supporting member 25. Further, 25b represents a notch through which the probe 10 penetrates. If the notch 25b exists, there is also an effect that it is possible to visually see a state of the one end 10a of the probe 10.

The first embodiment will be described below more specifically. The one end 10a of the probe 10 abuts against a predetermined portion of a biological soft tissue, and a portion of a tip end of the one end 10a bends from the abutted location such that pressing force or tensile force can be applied. The other end 10b of the probe 10 is detachably attached to the probe holder 11.

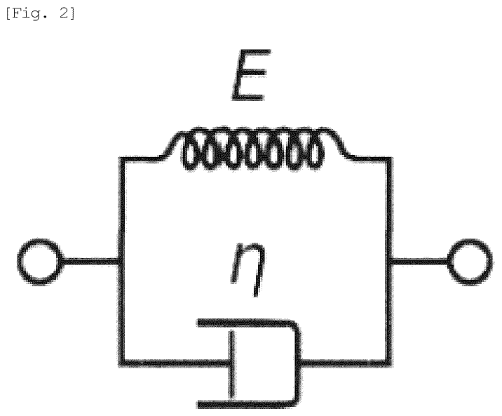

The biological soft tissue which is subjected to measurement is muscle, ligament, cartilage, skin or blood vessel, and the biological soft tissue is not especially limited only if the biological soft tissue is a biomedical tissue showing viscous elasticity. As shown in FIG. 2, the viscoelastic body has such characteristics that a spring E and a dashpot η are approximately arranged in parallel.

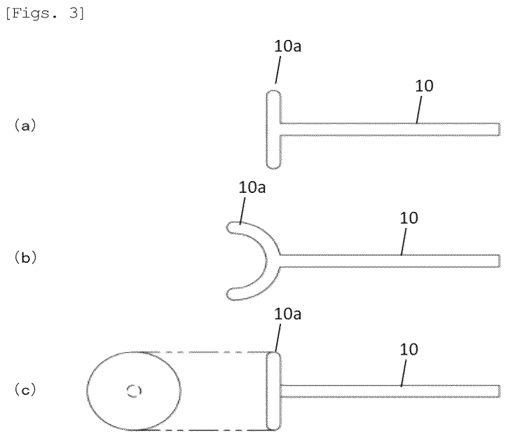

As material of the probe 10, material having excellent anticorrosion property such as stainless steel, other metal or resin is appropriately selected. Shape of the one end 10a of the probe 10 can appropriately be changed depending on the intended use. FIGS. 3(a) to 3(c) show example thereof. FIG. 3(a) shows T-shaped one end 10a having a portion extending in a direction perpendicular to Z-axis (axis in a long shaft direction of rod-shaped probe). FIG. 3(b) shows one end 10a bifurcated dichotomously. In the case of these shapes, it is possible to stably press ligament tissue. FIG. 3(c) shows disk-shaped one end 10a. In the case of the disk-shaped one end 10a, when cartilage tissue is pressed, it is possible to uniformly press a contact portion.

The probe holder 11 is connected to the load sensor 12 so that pressing force or tensile force applied to the probe 10 can be transmitted to the load sensor 12. In the first embodiment, three axes sensor (“MFS20-025” manufactured by JAPAN LINIAX CO., LTD. is used in the first embodiment) which can measure pressing force or tensile force not only in the long shaft direction (Z-axis) of the probe 10, but also in two axes (X-axis and Y-axis) perpendicular to Z-axis is used as the load sensor 12. Although it is possible to measure an analyte (biological soft tissue) only by means of the Z-axis of course, the analyte may be measured by means of a combination of two axes of Z-axis and X-axis, or Z-axis and Y-axis. Three axes are not always necessary, and the device may use one axis, two axes or three axes. However, when the probe 10 abuts against the analyte (biological soft tissue) obliquely, it is possible to correct this obliquity by obtaining resultant force of X-axis, Y-axis and Z-axis. Sensor outputs of X-axis, Y-axis and Z-axis of the load sensor 12 became analog input to the electronic circuit 21 in the body case 13 of the mechanical measuring device through the output terminal 12a.

The body case 13 is grasped by a user, and is composed of the bottomed cylindrical grasping portion 13a and the lid 13b which closes an opening of the grasping portion 13a. The probe 10 is mounted on one end of the grasping portion 13a, the one end is designed to such a size that the user (not shown) can easily grasp, and the other end of the grasping portion 13a is designed thicker than the one end. According to this, the user can stably operate the probe.

The stepping motor 14 is accommodated in the body case 13. The stepping motor 14 includes the motor coil 14a and the nut 14b, and the nut 14b extends along the Z-axial direction. The stepping motor 14 (Linear actuator 28F47-2.1-906 manufactured by Haydon Kerk Pitman is used in the first embodiment) rotates the nut 14b around its center axis by flowing electric current and energizing the motor coil 14a. According to this, the nut 14b functions as a rotor of the stepping motor 14. A thread groove is formed in an inner peripheral surface of the nut 14b along the Z-axis, and a screw thread is formed on an outer peripheral surface of the screw shaft 15. The screw thread is threadedly engaged with the thread groove. Hence, the screw shaft 15 can be moved in the Z-axis by rotating the nut 14b. This moving amount can be controlled by the pulse number or frequency applied to the stepping motor 14. It is necessary to set the moving speed in accordance with a kind of the biological soft tissue which is subjected to measurement, but in the stepping motor 14, it is possible to easily change the moving speed only by changing the pulse number or frequency. The assumed moving speed of the biological soft tissue which is subjected to measurement is 0.1 mm to 5 mm per second. BY the configuration shown in the first embodiment, the screw shaft 15 can be moved at a predetermined constant speed.

The spline shaft 17 is mounted on the screw shaft 15 through the connecting member 16. The screw shaft 15 moves in the Z-axial direction. As the screw shaft 15 moves in the Z-axial direction, the spline shaft 17 slides in the Z-axial direction along an inner portion of the ball spline 18. The ball spline 18 is mounted in a through hole of the lid 13b. According to this, the spline shaft 17 is supported by the ball spline 18 such that the spline shaft 17 can move along the Z-axial direction. One end of the spline shaft 17 in the Z-axial direction is mounted on the load sensor 12, and the load sensor 12 can be moved in the Z-axial direction at a predetermined constant speed.

The position detector 20 detects a moving distance of the connecting member 16 in the Z-axial direction by a position sensor (RDC1022A05 manufactured by ALPS CO., LTD. in the first embodiment). By this position detector 20, it is possible to detect the moving distance of the probe 10 in the Z-axial direction. In this case, the moving distance of the Z-axal direction may be detected by connecting an encoder to the stepping motor 14 instead of the position sensor.

The electronic circuit 21 drives and controls the stepping motor 14, the position detector 20 and the load sensor 12. The electronic circuit 21 is connected, through the external cable 23, to the controller 22 provided outside the body case 13.

The mechanical measuring device of the first embodiment measures the biological soft tissue. Therefore, an object which is subjected to measurement is soft and a reference probe which is necessary in the patent documents 2 and 4 cannot be used. This is because that when the reference probe is inserted into or pressed against a soft tissue, the object which is subjected to measurement does not have hard tissue which can receive its force. Even if the reference probe is brought into contact with the biological soft tissue and a measurement reference position is set, since the tissue is soft, a positional relation between the reference probe and the biological soft tissue is varied, and it is difficult to set the measurement reference position. In the mechanical measuring device of the fist embodiment, it is unnecessary to set the measurement reference position of the moving distance, and it is possible to measure without the reference position. The point when the load sensor 12 detects a load from the biological soft tissue can be regarded as a measurement starting point of the moving distance. Therefore, it is possible to measure a load applied to the biological soft tissue and a moving distance of the probe without using the reference probe.

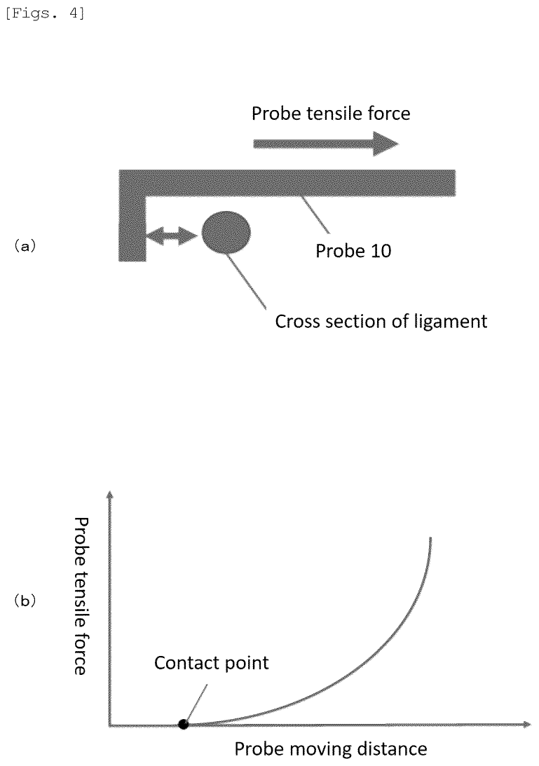

The first embodiment will be described in more detail using FIG. 4. FIG. 4(a) shows a positional relation between the probe 10 and the ligament (cross-section) before a measuring operation is started. In the mechanical measuring device of the first embodiment, when the probe 10 starts moving in the tensile force direction of the probe, the probe 10 is not in contact with the ligament which is subjected to the measurement, and as the probe moves in the tensile force direction, the probe 10 comes into contact with the ligament. After the probe 10 comes into contact with the ligament, measurement of a load applied to the ligament by the probe 10 is started, and measurement of the moving distance of the probe is also started. This measurement state is shown in FIG. 4(b). After the probe 10 comes into contact with the ligament, a relation between the moving distance and the load (tensile force) of the probe 10 is measured. Before this time point, even if the probe moves, the load remains zero. Therefore, it is unnecessary to set the measurement reference position by the reference probe, and the reference probe itself is unnecessary.

In the mechanical measuring device of the first embodiment, a user grasps the body case 13 with hands when the measuring operation is carried out. Therefore, the measuring state becomes unstable in some cases. Hence, the supporting member 25 is provided outside of the body case 13 (specific configuration is shown in FIGS. 1(b) and 1(c)), a tip end of the supporting member 25 is abutted against a portion of a surface of a measured object, and a body of the mechanical measuring device can be stabilized. The supporting member 25 can freely be extended and contracted in the Z-axial direction by adjusting the fixing screw 25a. However, the supporting member 25 is dispensable in the first embodiment. When there is no supporting member 25, movement of the probe 10 can automatically be driven by the stepping motor 14 by grasping the body case 13 in its static condition. Therefore, it is possible to measure the moving distance and the load. Hence, the supporting member 25 can be used as needed.

As described above, according to the first embodiment, (1) the probe 10 is moved at a constant speed by the stepping motor 14, and (2) the moving distance of the probe 10 is detected by the position detector 20. At the same time as (1) and (2), a load of the probe 10 can be measured in real time by the load sensor 12. Therefore, it is possible to precisely grasp the characteristics of the biological soft tissue.

### Relation Between Configuration and Circuit Configuration of Mechanical Measuring Device

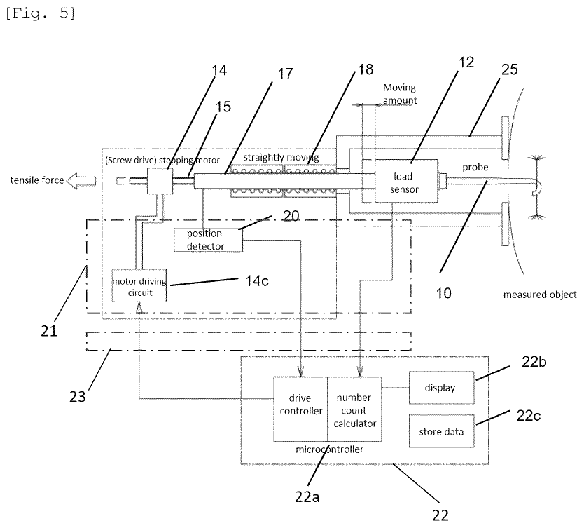

FIG. 5 is a schematic diagram showing a relation between mechanical measurement and a circuit configuration of the first embodiment. In FIG. 5, the same symbols in FIG. 1 show the same functions as those shown in FIG. 1.

As apparent from FIG. 5, in the first embodiment, output of the load sensor 12 and output of the position detector 20 are input to a microcomputer 22a of the controller 22. In the microcomputer 22a, a rigidity calculating operation for calculating rigidity (stress/distortion) of the biological soft tissue is carried out using measurement results of the load sensor 12 and the position detector 20, and a result thereof is output to a display means 22b and a recording means 22c. The microcomputer 22a controls a motor driving circuit 14c which controls a driving operation of the stepping motor 14. According to this configuration, it is possible to precisely control the moving speed of the probe 10.

### Entire Circuit Configuration

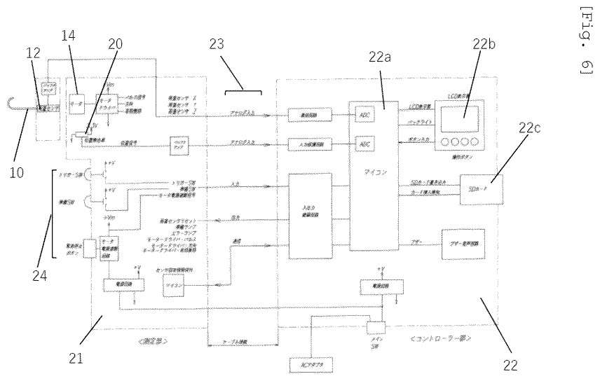

FIG. 6 shows a circuit configuration of the entire first embodiment. In FIG. 6, the same symbols in FIGS. 1 and 5 show the same functions as those shown in FIGS. 1 and 5. First, the electronic circuit 21 is placed in the body case 13, and the electronic circuit 21 controls the load sensor 12, the position detector 20 and the stepping motor 14. The controller 22 is provided outside of the body case 13 through the external cable 23.

The motor driving circuit 14c is provided for controlling the stepping motor 14. A control signal (pulse signal, signal direction, valid or invalid) is sent to the motor driving circuit 14c by the microcomputer 22a, and control is performed such that the probe 10 moves at a constant speed by the motor coil 14a of the stepping motor 14. More specifically, a pulse signal which becomes an energizing signal applied to the motor coil 14a from the motor driving circuit 14c is produced, and this energizing signal is applied to the motor coil 14a, thereby rotating the nut 14b. This rotation is controlled by applied pulse number. By changing forward and backward directions of applied pulse, it is possible to reversely rotate the nut 14b, and a rotation direction of the stepping motor 14 can be controlled such that a moving direction of the probe 10 is switched. By changing frequency of a clock signal of the motor driving circuit 14c, a rotation speed of the nut 14b is determined. As a result, a constant moving speed of the probe 10 is switched in a range of 0.1 mm to 5 mm per second.

Even if the moving speed of the probe 10 is not strictly constant, if the maximum value and the minimum value of the probe 10 are within a range of ±5% of the average value of the moving speed of the probe 10, it is possible to assume that the probe 10 is moving at a constant speed.

Further, a driving actuator is not limited to the stepping motor. It is only necessary that the driving actuator can control the speed, and a linear motor or a piston may be employed as the driving actuator.

Next, concerning control of the load sensor 12 and the position detector 20, an output signal from the load sensor 12 and an output signal (analogue signal) from the position detector 20 are sent to the controller 22 through the external cable 23.

Further, the mechanical measuring device of the first embodiment measures the biological soft tissue, prevents an abnormal operation of the device, and prevents a contingent medical error. Hence, a function which controls the operation switch 24 composed of a trigger SW, preparation SW and an emergency stopping SW is included in the electronic circuit 21 of the mechanical measuring device. When the emergency stopping SW is turned ON, a motor power supply shut-off circuit is operated, and an operation of the stepping motor 14 is emergently stopped by the controller 22.

The controller 22 is connected to the mechanical measuring device body through the external cable 23, the controller 22 processes signals from the load sensor 12, the position detector 20 and the stepping motor 14, and processes a signal for precisely grasping characteristics of the biological soft tissue. The controller 22 is composed of the microcomputer 22a, the display means 22b and the recording means 22c.

First, in the microcomputer 22a, an output signal of the load sensor 12 and an output signal from the position detector 20 are converted into digital values by a built-in analogue-digital conversing circuit. The microcomputer 22a calculates rigidity of the biological soft tissue based on the above-described digital values related to the moving distance, the pressing force or the tensile force of the probe 10.

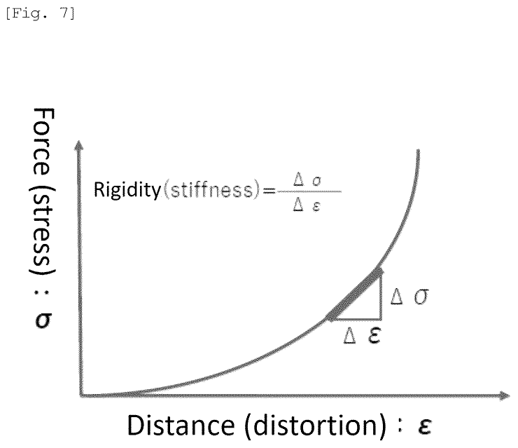

The rigidity of the biological soft tissue will be described using FIGS. 7 to 9. FIG. 7 is a diagram conceptually showing a relation between a distance (distortion) and force (stress) of the biological soft tissue. The moving distance of the probe 10 is represented by a distance ε, and pressing force or tensile force is represented by force σ. Firstly, a variation amount of force (load) applied to the probe 10 during a period from a “first time point” which is after the measurement operation is started to a “second time point” which is after the first time point is calculated as a variation amount Δσ of the force σ. Secondly, a variation amount of a position of the probe 10 during a period from the first time point to the second time point is calculated as a variation amount Δε of the distance (distortion) ε. Thirdly, the variation amount Δσ of the load applied to the probe 10 during the period from the first time point to the second time point is divided by a variation amount Δε of the position of the probe 10 from the first time point to the second time point, thereby obtaining rigidity (stiffness) Δσ/Δε at the second time point. Since this rigidity reflects characteristics of the biological soft tissue, it is considered that this value represents an evaluation determination index.

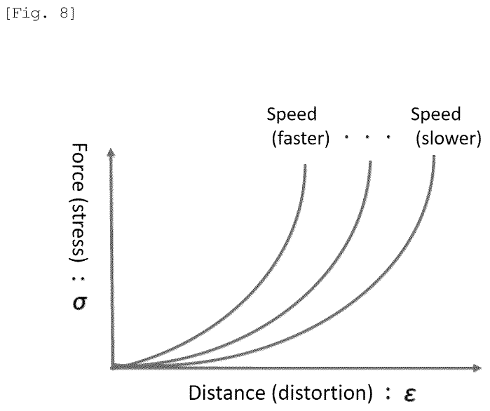

A relation between force and a distance has speed dependency. FIG. 8 is a schematic diagram showing the relation between the force and the distance when the force is applied to the same biological soft tissues at different speeds. As apparent from FIG. 8, different curved lines appear depending upon applied speeds. As the pressing force or the tensile force is greater, rising up of the force becomes faster. The biological soft tissues have inherent optimal speed, it is important to apply a load at the optimal speed which is inherent to the biological soft tissue and if not, precise measuring operation cannot be carried out. If speed at which the load is applied during one measurement operation is varied, it is not possible to precisely carry out the measuring operation neither. Therefore, during one measuring operation of rigidity, it is necessary to regularly keep the moving speed of the probe 10. Since the device shown in the first embodiment regularly keeps the moving speed of the probe 10, it is possible to precisely evaluate the characteristics of the biological soft tissue.

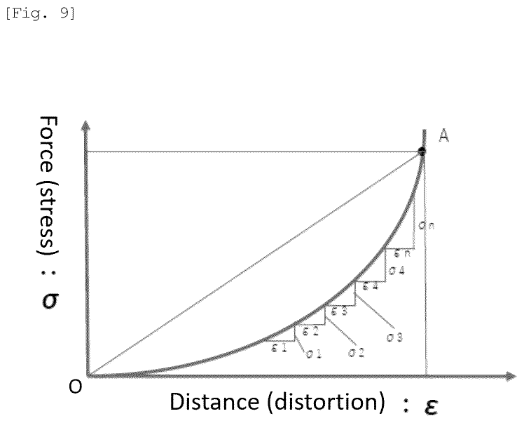

Characteristics of distortion and stress of the biological soft tissue show a curved line shape instead of a linear shape. Therefore, it cannot be said that measurement of rigidity at one location is precise. For example, the load is measured at the point in time when the load sensor 12 starts detecting a load applied to the biological soft tissue from the probe 10, and it is necessary to successively calculate the rigidity of the biological soft tissue at respective time points by measuring the moving distance of the probe 10 at the same time. FIG. 9 shows this calculating method. As shown in FIG. 9, the point in time when the load sensor 12 starts detecting a load applied to the biological soft tissue is set as a measuring-starting point. After the load sensor 12 starts detecting the load applied to the biological soft tissue from the probe 10, rigidity is repeatedly calculated and output at predetermined time intervals like Δσ1/Δσ1, Δσ2/Δε2. If rigidities at a plurality of points are obtained in this manner, it is possible to evaluate characteristics of the biological soft tissue more precisely, and optimal rigidity ασn/Δεn which is inherent to the measured object can be obtained. Since there are many kinds of measured objects, it is absolutely necessary to successively measure during movement of the probe 10 as described above. Since the device shown in the first embodiment can measure the moving distance and the load of the probe 10 in real time, such a measuring method can be realized. In the conventional examples shown in patent documents 1 to 4, if an attempt is made to calculate rigidity, only σ/ε which connects two points (i.e. a point O and a point A when the measuring operation is started as shown in FIG. 9) is measured, and Δσ1/Δε1, Δσn/Δεn at each time point of really necessary curved line shape cannot be obtained.

The display means 22b is electrically connected to the microcomputer 22a. The display means 22b displays the rigidity calculated by the microcomputer 22a, the load in the X-axis direction, the Y-axis direction and the Z-axis direction detected by the load sensor 12 and positional information from the position detector 20. A user can evaluate the biological soft tissue by reference to information displayed on the display means 22b.

The recording means 22c is connected to the microcomputer 22a, and data is written and taken out. Past accumulated data is stored in the recording means 22c. By comparing this accumulated data with a newly measured result, it is possible to quantitatively and objectively grasp the mechanical state of the biological soft tissue more precisely, and a user can determine the need of medical treatment such as operative treatment from this comparison result. By newly accumulating the measurement result in this recording means 22c, the accumulated data is enhanced.

Although it is not illustrated in the drawings, a communication module is provided, and it is possible to establish communication with an outside device (personal computer, tablet terminal or the like) from the communication module. Communication between the communication module and the outside device is performed by means of wire communication using LAN (Local Area Network) cable or serial communication cable. Communication between the communication module and the outside device may be performed by means of wireless communication using WiFi (registered trademark) or Bluetooth (registered trademark), and calculated data may be output through the communication module.

### Measurement Result.

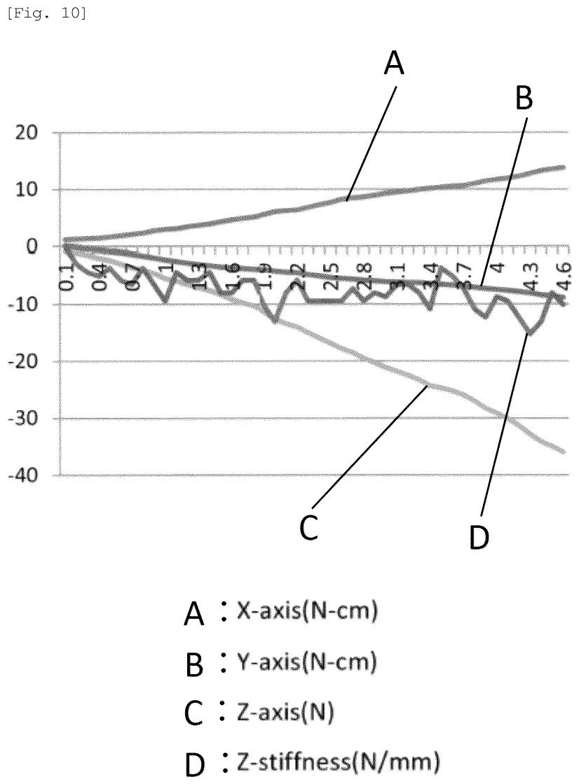

FIG. 10 shows a display example of the display means 22b of the mechanical measuring device according to the first embodiment when a ligament of a pig is measured as one example. Here, analogue display is performed but digital display can be performed if required. In FIG. 10, A represents moment (N-cm) in the X-axial direction, B represents moment (N-cm) in the Y-axial direction, C represents a load (N) in the Z-axis direction, and D represents a rigidity value (stiffness) (N/nm). These values are calculated by the microcomputer 22a based on outputs of the load sensor 12 and the position detector 20. The rigidity value D is calculated every moving distance of 0.1 mm. Numeric values shown in the drawing are expressed dimensionlessly.

Here, A and B represent moments, loads applied to the tip end of the probe in the X-axis direction and Y-axis direction can be calculated from these values, and the load in the Z-axis direction can be corrected using these values. The rigidity value D is varied vertically in the graph, but this is because a width Δε of the moving distance is as small as 0.1 mm in this case, and if the width is made greater, the variation is reduced, and the rigidity value comes close to a value near a real state. The values A, B, C and D are detected continuously from the measurement starting time point (i.e. from a zero point of moving distance) and synchronously to each other, and a rigidity value at an arbitrary point of the moving distance can be obtained. It is possible to obtain a rigidity value at a moving distance which is optimal for expressing physical property of the measured object.

### Second Embodiment

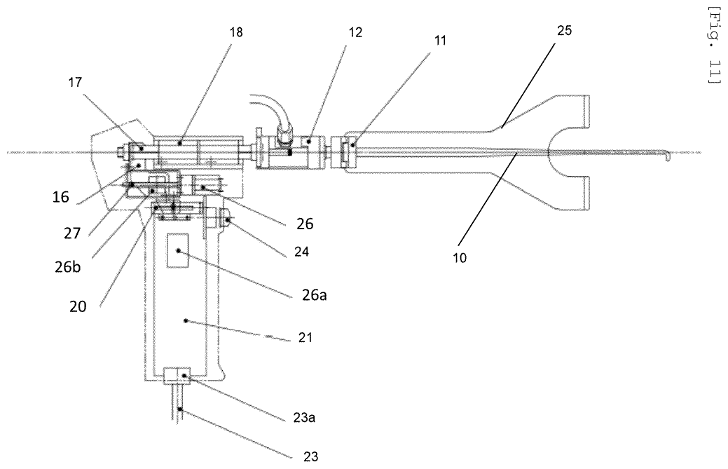

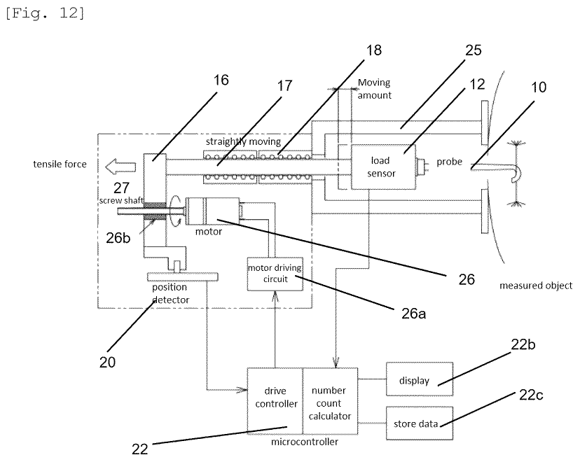

FIG. 11 is a sectional view of a structure of a mechanical measuring device according to a second embodiment, and FIG. 12 is a schematic diagram showing a relation between the structure of the second embodiment and a circuit configuration thereof. In the second embodiment, the same symbols as those of the first embodiment show the same functions as those shown in FIGS. 1, 5 and 6.

The second embodiment is different from the first embodiment in that a pistol structure like an electric drill of a machine tool is employed. In FIGS. 11 and 12, 26 represents a DC gear motor, 26a represents a motor driving circuit, 26b represents a nut, and 27 represents a screw shaft. Here, a thread groove is formed in an inner peripheral surface of the nut 26b along the Z-axial direction, and a screw thread which is threadedly engaged with this thread groove is formed on an outer peripheral surface of the screw shaft 27. As the screw shaft 27 rotates, the connecting member 16 moves in the Z-axial direction. Here, since the screw shaft 27 of the DC gear motor 26 is placed in parallel to the spline shaft 17, a length of the body case 13 becomes long in the vertical direction of the drawings, and an electric drill structure is realized.

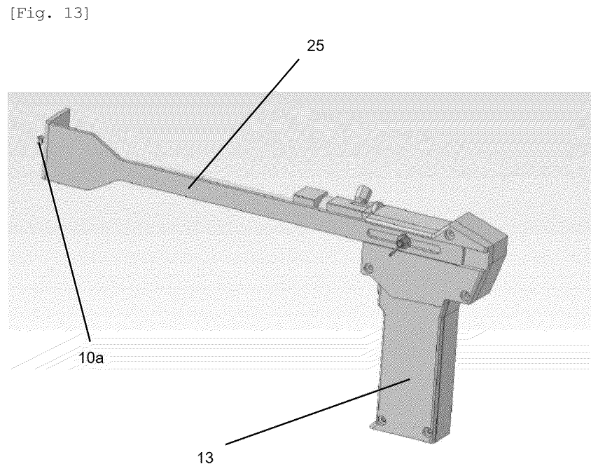

FIG. 13 is a perspective view of the mechanical measuring device according to the second embodiment. A user grasps a handgrip of the body case 13 of the mechanical measuring device to use. The supporting member 25 shown here is not provided with the notch 25b.

### Third Embodiment

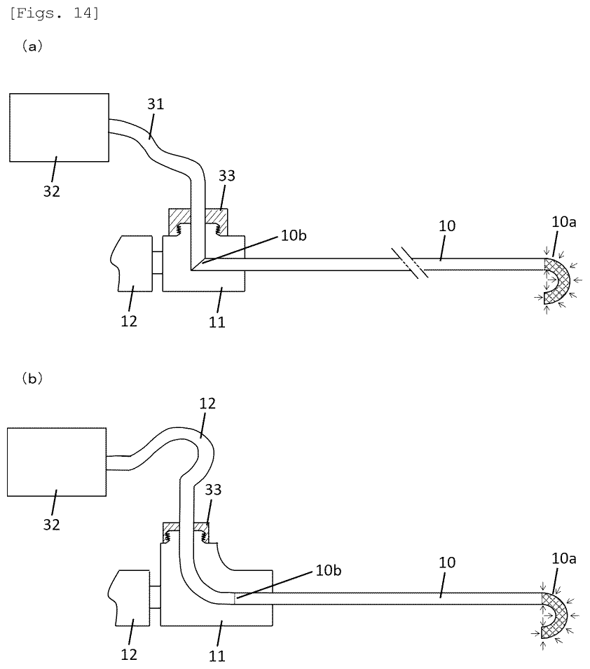

FIG. 14 are sectional views of a structure of an essential portion of a mechanical measuring device of a third embodiment. The structure shown in FIG. 14 can be applied to the first and second embodiments, and the first and second embodiments can be applied to a structure which is not shown in FIG. 14. In the third embodiment also, the same symbols are allocated to the same functional members as those of the first and second embodiments, and description thereof will be omitted.

In the third embodiment, the probe 10 is made of translucent material, light is introduced from the other end 10b of the probe 10, and scattering light is emitted by the one end 10a of the probe 10. For example, acrylic, polycarbonate and other transparent hard material are suitable for the probe 10.

A surface of the one end 10a of the probe 10 is subjected to pearskin finish. According to this, light can be scattered outside. An emitting range of the scattered light may be entire periphery of the one end 10a, or may be an inner scope of a curved portion.

In order to introduce light into the probe 10, an optical fiber 31 is connected to the other end 10b of the probe 10. Light is supplied to the optical fiber 31 from an optical fiber light source 32.

An end of the optical fiber 31 on the side of the probe 10 is fixed to the probe holder 11 by a set screw 33.

FIG. 14(a) shows a case in which an axis of the optical fiber 31 and an axis of the other end 10b of the probe 10 are connected to each other such that these axes are deviated by predetermined angle, and FIG. 14(b) shows a case in which the axis of the optical fiber 31 and the axis of the other end 10b of the probe 10 are connected to each other such that these axes coincide with each other.

When the one end 10a of the probe 10 abuts against a ligament for example, it is necessary to check if the one end 10a reliably captures the ligament, but since the ligament exists in a joint, it is relatively dark and it is difficult to check the ligament. By emitting the scattered light by the one end 10a of the probe 10 as in this embodiment, it is possible to check the abutment state between the one end 10a of the probe 10 and the ligament.

By scattering light to outside from the bent one end 10a as in this embodiment, it is possible to lighten the periphery of the one end 10a, and it is possible to observe the state of an affected part of the soft tissue.

### Fourth Embodiment

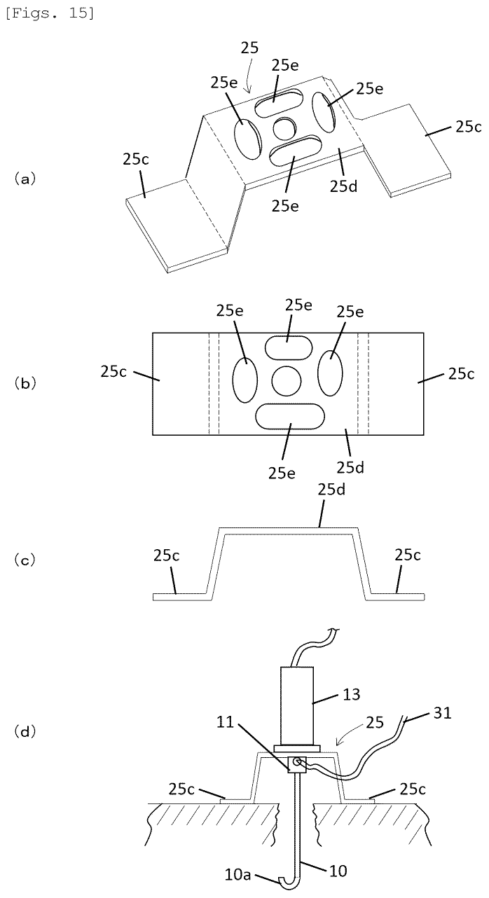

FIG. 15 are explanatory diagrams of a supporting member used in a mechanical measuring device according to a fourth embodiment, wherein FIG. 15(a) is a perspective view of the supporting member, FIG. 15(b) is a top view of the supporting member, FIG. 15(c) is a side of the supporting member, and FIG. 15(d) is a side view showing a using state of the supporting member. The supporting member shown in FIG. 15 can be applied instead of the supporting members of the first and second embodiments. In the fourth embodiment also, the same symbols are allocated to the same functional members as those of the first to third embodiments, and description thereof will be omitted.

The supporting member 25 of the fourth embodiment includes a pair of abutment pieces 25c which abut against a portion of a surface of a measured object (human body), a probe holder holding portion 25d formed between the pair of abutment pieces 25c, and observation windows 25e formed in the probe holder holding portion 25d.

The supporting member 25 of the fourth embodiment is separated from the body case 13 having the probe 10, and the supporting member 25 can be placed on a measured object.

By using the supporting member 25 which is separated from the body case 13 having the probe 10 in this manner, the supporting member 25 can be used in accordance with a shape and a state of the measured object.

The embodiments disclosed here are shown as examples in every respect, the present invention is not limited to the embodiments, basic scopes of this disclosure are shown in patent claims not in the embodiments, and it is intended that all of changes within the patent claims, equivalent meanings and scopes are included. The biological soft tissues are described in the above-described embodiments, but the invention is not limited to the biological soft tissue and the invention can effectively be used also for material showing viscous elasticity (e.g. foods, rubber products and other industrial materials).

### INDUSTRIAL APPLICABILITY

The embodiments are advantageously applied especially for mechanical measuring of a biological soft tissue.

### EXPLANATION OF SYMBOLS

10 probe
10a one end
10b other end
11 probe holder
12 load sensor (load measuring means)
12a output terminal
13 body case
13a grasping portion
13b lid
14 stepping motor (moving means)
14a motor coil
14b nut
14c motor driving circuit
15 screw shaft
16 connecting member
17 spline shaft
18 ball spline
19 cover
20 position detector (distance measuring means)
21 electronic circuit
22 controller
22a microcomputer
22b display means
22c recording means
23 external cable
24 operation switch
25 supporting member (supporting means)
25a fixing screw
25b notch
25c abutment pieces
25d probe holder holding portion
25e observation window
26 DC gear motor
26a motor driving circuit
26b nut
27 screw shaft
31 optical fiber
32 optical fiber light source
33 set screw

1: A mechanical measuring device comprising a body case, a probe extending from the body case and abutting against a predetermined portion of a biological soft tissue, a load measuring means which is integrally connected to the probe and which measures a load applied to the biological soft tissue, a moving means for moving the probe at a constant speed, and a distance measuring means for measuring a moving distance by the moving means, wherein the load applied to the biological soft tissue and the moving distance of the probe are simultaneously measured.

2: The mechanical measuring device according to claim 1, further comprising rigidity calculating means for calculating rigidity (force/distance) of the biological soft tissue by using output of the load measuring means and output of the distance measuring means.

3: The mechanical measuring device according to claim 1, wherein a measuring operation of the moving distance of the probe is started from point in time when the load measuring means starts measuring the load applied to the biological soft tissue.

4: The mechanical measuring device according to claim 1, wherein the constant speed by the moving means is appropriately changed.

5: The mechanical measuring device according to claim 1, further comprising display means for displaying output of the load measuring means, output of the distance measuring means and output of the rigidity calculating means, and recording means for recording the outputs.

6: The mechanical measuring device according to claim 1, further comprising supporting means for fixing a position of the body case.

7: The mechanical measuring device according to claim 1, wherein the probe is made of translucent material, and scattering light is emitted from one end of the probe.
